(19) United States
(12) Patent Application Publication
(10) Pub. No.: US 2025/0195400 A1
(43) Pub. Date: Jun. 19, 2025

(54) PIGMENT DISPERSIONS WITH HYDROPHOBIC POLYMER

(71) Applicant: L'OREAL, Paris (FR)

(72) Inventors: Xuefei LIU, Edison, NJ (US); Ritesh Kumar SINHA, Linden, NJ (US); Hy Si BUI, Piscataway, NJ (US)

(73) Assignee: L'OREAL, Paris (FR)

(21) Appl. No.: 18/544,312

(22) Filed: Dec. 18, 2023

(51) Int. Cl. A61K 8/92; A61K 8/06; A61Q 1/00

(52) U.S. Cl. CPC A61K 8/922; A61K 8/062; A61Q 1/00; A61K 2800/412; A61K 2800/43

(57) ABSTRACT

The instant disclosure describes pigment dispersions useful as cosmetics and also useful in the manufacture of cosmetics. The pigment dispersions include: (a) a hydrophobic polymer formed as a reaction product of a natural or food-derived oil and a methacrylate or acrylate polymer; (b) one or more solvents capable of dissolving the hydrophobic polymer; and (c) particles of one or more pigments dispersed throughout the one or more solvents. Methods of making and using the pigment dispersions are also described.

### FIELD OF THE DISCLOSURE

The instant disclosure is drawn to pigment dispersions, cosmetic compositions comprising the pigment dispersions, and methods for making them.

### BACKGROUND

Products are often colored with coloring agents, including pigments. Pigments are used across many industries, including the cosmetic industry. A pigment is a substance that imparts color to another substance or material to which it is added. Pigments provide color through selective absorption and reflection of certain wavelengths of light. Many pigments, such as inorganic pigments, are generally insoluble in most liquid vehicles, such as organic solvents and water. This poses formulation challenges and limitations on their use.

Various cosmetic products, such as loose or compact powders, make-up foundations, blushes, eye shadows, and lipsticks are colored using inorganic pigments dispersed in a carrier. Examples of these inorganic pigments are iron oxides, zinc oxide, talc, titanium dioxide, chromium hydroxide, and chromium oxide. The cosmetic products that include the inorganic pigments are formulated as oil-in-water emulsions, water-in-oil emulsions, or anhydrous compositions. Inorganic pigments are often provided as a dry powder that is incorporated into an acceptable carrier or medium. Incorporating the dry powder often requires a substantial amount time to ensure the pigments are uniformly dispersed. Even if initially the inorganic pigments eventually become uniformly dispersed, they can agglomerate, or phase separate over time. Particles of inorganic pigments can attract one another and form a colloid or enlarged clump of pigment, especially when added directly to oil-in-water dispersions.

A variety of methods have been attempted to enhance dispersion and prevent agglomeration of pigments. For example, the particles of pigments can be hydrophobically treated and directly added to a carrier, including an oil-in-water emulsion. A common problem, however, is the lack of consistency and evenness. Due to their magnetic nature, various inorganic pigments agglomerate even if they have been surface-treated. Thus, to keep the pigments evenly dispersed high-speed shearing mixers are used. Even when such techniques are implemented, re-agglomeration may occur in holding tanks before the finished product is poured into retail sale packaging.

To prevent this, many manufacturers add a second high-speed mixer to the holding tank. In some cases, however, a high-speed mixer over-agitates, whips, or froths the product before it is poured into retail containers. Resulting froth or air bubbles can cause color changes, discoloration, air pockets and/or pigment particle agglomeration in the final product.

Colloidal stability is a fundamental property of pigment dispersion that determines the particles' resistance to flocculation, aggregation, and sedimentation during preparation, storage, and application. In general, colloidal stability depends upon the balance of the repulsive and attractive forces that exist between particles as they approach one another in liquid media. If the particles have a mutual repulsion and resistance to gravitational forces, then the dispersion will typically remain relatively stable. However, if attractive forces prevail, then some instability mechanism will eventually take place, for example flocculation, aggregation, flotation, and sedimentation. As a result, the dispersion will become unusable. Moreover, colloidal stability determines essential properties of pigment dispersions such as coloristics, rheological behavior, mobility of pigment particles, and particle size distribution.

While various strategies have been attempted to disperse and use pigments in cosmetic products, there remains a need for stable dispersions of pigments, efficient and simple methods for generating the dispersions, and cosmetic products that incorporate them.

### SUMMARY OF THE DISCLOSURE

The present disclosure is drawn to pigment dispersions useful for cosmetic and personal care products. The dispersions include particles of one or more pigments and a unique hydrophobic polymer, solubilized in a solvent. The inventors found that the unique hydrophobic polymer surprisingly reduces agglomeration and enhances stability, uniformity, and texture of the dispersions. The pigment dispersions are themselves useful as cosmetic products but can also be used as a component or ingredient of further cosmetic products. For example, the inorganic pigment dispersions can form an oil phase of an emulsion, i.e., an oil-in-water emulsion, wherein the oil-in-water emulsion functions as the cosmetic product. Regardless, the pigment dispersions are surprisingly versatile, robust, and easy to manufacture and use.

The pigment dispersions typically include:
(a) a hydrophobic polymer formed as a reaction product of a natural or food-derived oil and a methacrylate or acrylate polymer;
(b) one or more solvents capable of solubilizing the hydrophobic polymer of (a); and
(c) particles of one or more inorganic pigment dispersed through the one or more solvents of (b).

The average particle size of the pigments is not particularly limited. Different particle sizes often have different visual effects and properties and therefore different applications may require different particles sizes. Nonetheless, in various embodiments, the average particle size of the pigments is typically less than 15 μm, or from about 100 nm to about 10 μm.

The pigment dispersions are typically anhydrous or essentially anhydrous. The one or more solvents capable of solubilizing the hydrophobic polymer form the carrier phase of the pigment dispersions. The inorganic pigment dispersions can themselves function as a cosmetic product, for example a makeup product. Due to their versatility, stability, and ease of use, the pigment dispersions can also be used as a component or ingredient in the manufacture of further cosmetic products. For example, the pigment dispersion may form an oil phase of an oil-in-water emulsion and the oil-in-water emulsion may function as a cosmetic product.

The hydrophobic polymer is a reaction product of a natural or food-derived oil and an acrylate or methacrylate polymer. According to embodiments of the disclosure, however, the hydrophobic polymer is the reaction product of a natural or food-derived oil and a methacrylate polymer. The natural or food-derived oil may be a drying oil or a semi-drying oil. Nonlimiting examples include linseed oil, sunflower oil, tung oil, fish oil, cottonseed oil, soybean oil, or combinations thereof. The methacrylate polymer can be formed from methacrylate monomers, for example, monomers selected from isobutyl methacrylate, methyl methacrylate, ethyl methacrylate, n-butyl methacrylate, and combinations thereof. In a preferred embodiment, the hydrophobic polymer is formed from a natural or food-derived oil and an isobutyl methacrylate polymer.

In various embodiments, the hydrophobic polymer is the reaction product of about 50 to about 85 parts by weight of the natural or food-derived oil and about 15 to about 50 parts by weight of the methacrylate or acrylate polymer. More specifically, the hydrophobic polymer may be the reaction product of about 72 to about 77 parts by weight of the natural or food-derived oil and about 23 to about 28 parts by weight of a methacrylate polymer. For example, the hydrophobic polymer may be the reaction product of linseed oil and poly(isobutyl methacrylate) in a suitable solvent, such as, for example, 2,2,4-trimethyl-1,3-pentanediol monoisobutyrate. Preferably, the reaction product is formed from about 72 to about 77% of linseed oil and about 23 to about 28% of isobutyl methacrylate polymer in a suitable solvent, such as 2,2,4-trimethyl-1,3-pentanediol monoisobutyrate.

One or more solvents capable of solubilizing the hydrophobic polymer of (a) may be used. A single solvent may be used, or a combination of solvents may be used, wherein the combination of solvents can solubilize the hydrophobic polymer. In various embodiments, the one or more solvents capable of solubilizing the hydrophobic polymer have a dispersion component (D), a polar component (P), and a hydrogen bonding component (H), and a distance (Ra) per Hansen Solubility Parameter or less than or equal to 13.4 MPa^{0.5}, wherein (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

Nonlimiting examples of solvents capable of solubilizing the hydrophobic polymer include polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof. Polycitrnoellol acetate is particularly useful. Caprylic/capric triglyceride is also particularly useful. Combinations of solvents are often used.

Nonlimiting examples of inorganic pigments include colored metal oxides, metal hydroxides, metal oxide hydrates, silicates, metal sulfides, complex metal cyanides, metal sulfates, bronze pigments, colored mica- or mica-based pigments coated with at least one metal oxide and/or a metal oxychloride, and combinations thereof. The one or more inorganic pigments may be surface-modified, for example, hydrophobically surface-modified. However, surface modification is not necessary. More specific but nonlimiting examples include unmodified or surface-modified inorganic pigments selected from activated carbon, talc, iron oxides, titanium dioxide, zinc oxide, silica, cerium oxides, zirconium oxides, aluminum oxides, calcium carbonate, barium sulfate, chromium oxides, manganese oxides, bismuth oxychloride, ultramarine, calcium sulfate, alkali magnesium silicates or mixtures thereof.

The pigment dispersions can themselves form a cosmetic product or can be further processed or incorporated into a cosmetic product. Accordingly, the pigment dispersions may optionally include additional ingredients such as one or more film forming polymers, thickening agents, active ingredients including skin active ingredients, miscellaneous ingredients, or combinations thereof. In various embodiments, the pigment dispersion is combined with an aqueous phase to form an emulsion, preferably an oil-in-water emulsion. The pigment dispersions and compositions comprising them form cosmetic and personal care compositions.

### BRIEF DESCRIPTION OF THE DRAWINGS

Implementation of the present technology is described, by way of example only, with reference to the attached figures, wherein:

FIG. 1 shows magnified (10×) pictures of inventive and comparative dispersions according to the instant disclosure.

FIG. 2 shows magnified (10×) pictures of an inventive and a comparative dispersion according to the instant disclosure.

It should be understood that the various aspects are not limited to the arrangements and instrumentality shown in the drawings.

### DETAILED DESCRIPTION OF THE DISCLOSURE

The present disclosure is drawn to pigment dispersions that themselves are useful as cosmetic products. In addition, the pigment dispersions can be combined with additional elements or ingredients to formulate a cosmetic product, include an aqueous phase. The pigment dispersions include particles of one or more pigments that become stably dispersed due in part to the use of a unique hydrophobic polymer. The unique hydrophobic polymer is solubilized in one or more solvents and carries the one or more inorganic pigments, which are dispersed throughout the one or more solvents. The inventors surprisingly discovered that the unique hydrophobic polymer reduces agglomeration of the pigments, and enhances the stability, uniformity, and texture of the pigment dispersion. The pigment dispersions typically include:
(a) a hydrophobic polymer formed as a reaction product of a natural or food-derived oil and a methacrylate or acrylate polymer;
(b) one or more solvents capable of solubilizing the hydrophobic polymer of (a); and
(c) particles of one or more pigments dispersed throughout the one or more solvents of (b).

### (a) Hydrophobic Polymer

The hydrophobic polymer is a reaction product of a natural or food-derived oil (oil component) and an acrylate component. In particular, the natural or food-derived oil may be a drying oil, preferably linseed oil. The reaction product may include an isobutyl methacrylate backbone with a plurality of linseed oil side chains. Preferably, the reaction product is a product sold under the MYCELX® brand from MYCELX Technologies Corporation of Gainesville, Georgia. See U.S. Pat. No. 5,698,139 for a description of MYCELX materials, which is incorporated herein by reference in its entirety.

The hydrophobic polymer is comprised of an oil component and a polymer component, typically reacted in a solvent. In a preferred embodiment, the hydrophobic polymer is a reaction product of linseed oil and poly(isobutyl methacrylate), in a solvent such as 2,2,4-trimethyl-1,3-pentanediol-monoisobutyrate.

The oil component is derived from glycerin and carboxylic acids, such as linseed fatty acid to form monoglycerides, diglycerides, and triglycerides. The oil component is preferably derived from plant/vegetable or natural origin. Vegetable oils are obtained by cold pressing the seeds of a plant to obtain the oil contained therein. Of the vegetable oils, drying oils such as linseed and tung oil, semi-drying oils such as soybean and cotton seed oil, and non-drying oils such as coconut oil may be used as the oil component. The oil component typically forms about 72% to 77%, or most preferably, 74.62%, of the hydrophobic polymer (e.g., linseed oil/isobutyl methacrylate).

The polymer component may be derived from α and β-unsaturated carbonyl compounds. The polymer component is the resultant product of a monomer which is an ester of an acrylic acid, crotonic acid, isocrotonic acid, methacrylic acid, sorbic acid, cinnamic acid, maleic acid, fumaric acid, and methyl methacrylic acid. Nonlimiting examples of useful polymers which cover any number of reaction possibilities between the esters of such compounds include acrylate polymers, methyl methacrylate polymers, methyl/n-butyl methacrylate polymers, methacrylate copolymers, ethyl methacrylate, n-butyl methacrylate, isobutyl methacrylate, n-butyl/isobutyl methacrylate copolymers, or combinations thereof.

Preferably the polymer is poly(isobutyl methacrylate) and the hydrophobic polymer is poly(linseed oil/isobutyl methacrylate). The hydrophobic polymer is a reaction product typically formed in a liquid solvent able to dissolve or dilute the polymer component and the formed hydrophobic polymer (poly(oil/polymer)). The solvent, or diluent should generally comprise any liquid or mixture of liquids that is able to dissolve or dilute the polymer component and the formed hydrophobic polymer. The solvent/diluent can control the evaporation, desired flow, and coalescing of the hydrophobic polymer. The solvent may be, for example, an aliphatic hydrocarbon, aromatic hydrocarbon, alcohols, ketones, ethers, aldehydes, phenols, carboxylic acids, carboxylates, synthetic chemicals and naturally occurring substances. Preferably the solvent is 2,2,4-trimethyl-1,3-pentanediol-monoisobutyrate. Hydrophobic polymers according to the instant disclosure and methods for making them are described, for example, in U.S. Pat. Nos. 5,437,793, 5,698,139, 5,837,146, 5,961,823, 6,180,010, 6,475,393, and 6,805,727, which are incorporated herein by reference in their entireties. The preferred hydrophobic polymer may be designated as poly(linseed oil/isobutyl methacrylate).

The amount of hydrophobic polymer in the pigment dispersion will vary but is typically in an amount from about 0.1 to about 30 wt. %, based on the total weight of the pigment dispersion. In further embodiments, the pigment dispersion includes from about 0.1 to about 20 wt. %, about 0.1 to about 15 wt. %, about 0.1 to about 10 wt. %, about 0.1 to about 5 wt. %, about 0.5 to about 30 wt. %, about 0.5 to about 20 wt. %, about 0.5 to about 15 wt. %, about 0.5 to about 10 wt. %, about 0.5 to about 5 wt. %, about 1 to about 30 wt. %, about 1 to about 20 wt. %, about 1 to about 15 wt. %, about 1 to about 10 wt. %, about 1 to about 5 wt. %, about 0.1 to about 4 wt. %, or about 0.5 to about 3 wt. %, based on the total weight of the pigment dispersion.

### (b) Solvent Capable of Solubilizing (a)

The oil phase of the pigment dispersion includes the hydrophobic polymer of (a) dissolved in one or more solvents capable of solubilizing the hydrophobic polymer. The one or more solvents may include a solvent used to in the reaction to form the hydrophobic polymer, e.g., 2,2,4-trimethyl-1,3-pentanediol-monoisobutyrate. The one or more solvents may be a single solvent or a plurality of solvents. For example, in various embodiments, solvents capable of solubilizing the hydrophobic polymer of (a) have a dispersion component (D), a polar component (P), a hydrogen bonding component (H), and a distance (Ra) of less than or equal to 13.4 MPa^{0.5} per the Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

In a preferred embodiment, the one or more solvents have a dispersion component (D), a polar component (P), a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}.

The solvent may be an oil. The term “oil” is intended to mean a non-aqueous compound, non-miscible in water, liquid at 25° C. and atmospheric pressure (760 mmHg; 1.013×10⁵ Pa). The solvent may be a non-silicone oil (e.g., an oil that does not contain silicon atoms, and in particular does not contain Si—O groups). Non-limiting examples of the one or more solvents include caprylic/capric triglyceride, isopropyl myristate, polycitronellol acetate, and combinations thereof. The solvent may include acetone. The solvent may include oleic acid. The solvent may include an oleic acid containing oil (such as a vegetable oil). Table 1, below, shows values of D, P, and H, as well as Ra for the allowable ranges as well as preferred ranges, for several solvents.

TABLE 1

| | | | | Ra | Ra |
|---|---|---|---|---|---|
| | | | | (Allowable | (Preferred |
| Solvent (b) | D | P | H | Range) | Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl | 15.9 | 2.1 | 2.8 | 10.70 | 9.41 |
| Myristate | | | | | |
| Isopropyl Alcohol | 15.8 | 6.1 | 6.4 | 7.02 | 5.54 |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol | 16.4 | 3 | 4.2 | 9.02 | 7.76 |
| Acetate | | | | | |
| Acetone | 15.5 | 10.4 | 7 | 8.61 | 7.38 |
| Oleic Acid | 16 | 2.8 | 6.2 | 7.27 | 5.98 |

In some embodiments, if oleic acid is utilized, at least some of the oleic acid may be provided by a vegetable oil. The vegetable oil may be a seed or nut oil. The vegetable oil may have an oleic acid content of at least 20% by weight of the vegetable oil. The vegetable oil may include sunflower oil, soybean oil, macadamia nut oil, and/or avocado oil. In some embodiments, the composition may include macadamia nut oil, and may be free, or substantially free, of other vegetable oils.

For purposes of the instant disclosure, the one or more of the solvents capable of solubilizing the hydrophobic polymer of (a) may not individually solubilize the hydrophobic polymer but when combined with other solvents, the combination solubilizes the hydrophobic polymer. Thus, when referring to a total amount of one or more solvents capable of solubilizing the hydrophobic polymer of (a), the inclusion of all solvents that in combination solubilize the hydrophobic polymer of (a) is intended, even if one or more solvents in the combination do not individually solubilize the hydrophobic polymer of (a).

Nonlimiting examples of solvents useful for solubilizing the hydrophobic polymer of (a), individually or in combination with other solvents, include polycitronellol acetate, caprylic/capric triglyceride, isododecane, isohexadecane, tetradecane, isopropyl myristate, octyldodecanol, ethanol, phenoxyethanol, castor oil, and mixtures thereof. In a preferred embodiment, at least one of the one or more solvents capable of solubilizing the hydrophobic polymer are selected from caprylic/capric triglyceride, polycitronellol acetate, isododecane, and mixtures thereof. In another preferred embodiment, at least one of the one or more solvents capable of solubilizing the hydrophobic polymer is polycitronellol acetate, caprylic/capric triglyceride, or a combination thereof.

Nonlimiting solvents that individually or in combination with other solvents are useful for solubilizing the hydrophobic polymer of (a) include dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof.

The total amount of the one or more solvents capable of solubilizing the hydrophobic polymer of (a) in the pigment dispersion will vary and depends on amounts of other components of the dispersion, for example, the amount of pigment. Nonetheless, the total amount of the one or more solvents capable solubilizing the hydrophobic polymer can be from about 10 wt. % to about 90 wt. %, based on the total amount of the dispersion. In further embodiments, the total amount of the one or more solvents capable of solubilizing the hydrophobic polymer is from about 10 wt. % to about 80 wt. %, about 10 to about 70 wt. %, about 10 to about 60 wt. %, about 10 to about 50 wt. %, about 20 to about 90 wt. %, about 20 to about 80 wt. %, about 20 to about 70 wt. %, about 20 to about 60 wt. %, about 20 to about 50 wt. %, about 30 to about 90 wt. %, about 30 to about 80 wt. %, about 30 to about 70 wt. %, about 30 to about 60 wt. %, about 30 to about 50 wt. %, about 40 to about 90 wt. %, about 40 to about 80 wt. %, about 40 to about 70 wt. %, about 40 to about 60 wt. %, about 50 to about 90 wt. %, about 50 to about 80 wt. %, about 50 to about 60 wt. %, about 60 to about 90 wt. %, about 60 to about 80 wt. %, about 60 to about 70 wt. %, about 70 to about 90 wt. %, about 70 to about 80 wt. %, based on the total weight of the pigment dispersion.

In further embodiments, when the amount of pigment is low, the total amount of the one or more solvents capable of solubilizing the hydrophobic polymer can be higher, for example, from about 70 to about 99 wt. %, based on the total weight of the pigment dispersion. Along these lines, the total amount of the one or more solvents capable of solubilizing the hydrophobic polymer may be from about 75 to about 99 wt. %, about 80 to about 99 wt. %, about 85 to 99 wt. %, about 70 to about 95 wt. %, about 75 to about 95 wt. %, about 80 to about 95 wt. %, about 85 to about 95 wt. %, about 70 to about 90 wt. %, about 75 to about 90 wt. %, about 80 to about 90 wt. %, based on the total amount of the pigment dispersion.

### (c) Pigments

As used herein, the term “pigment” includes both organic and inorganic pigments. Pigments are differentiated from dye due to their lack of solubility, especially in water. Pigments are insoluble in most media, especially inorganic pigments. Dyes, one the other hand, are typically soluble in water. Organic pigments are synthetic materials based on carbon and are often derived from petrochemicals. They generally are not stable at elevated temperatures and have partial solubility in strong solvents, but do not dissolve in water. Inorganic pigments (sometimes referred to as “mineral pigments”) include metal salts, oxides, and minerals, some natural and some synthetic, that are generally are stable at elevated temperatures and do not dissolve in solvents. Due to their stable chemical structures, most inorganic pigments have better weatherability, dispersibility and opacity than organic pigments, however they will typically have lower chromaticity and tinctorial strength. Organic pigments tend to be more vibrant and have a wider range of colors, while inorganic pigments are more muted and earthy.

Any number of pigments of any number of primary or secondary colors, or black or white, for example, may be used. As specific examples, the pigment may be any color, including, as examples, a cyan pigment, a magenta pigment, a yellow pigment, a black pigment, a violet pigment, a green pigment, a brown pigment, an orange pigment, a purple pigment, a white pigment, or combinations thereof.

### Inorganic Pigments

Inorganic pigment may optionally be surface-modified, for example, hydrophobically surface-modified but surface modification is certainly not necessary. Nonlimiting examples of inorganic pigments include, but are not limited to, metal oxides, metal borates, metal sulfates, metal sulfides, metal chromates, metal carbonates, metal selenides, and combinations thereof. In some embodiments, suitable inorganic pigments can include, for example, titanium (IV) oxide, iron (III) oxide, zinc oxide, or combinations thereof. Other inorganic pigments that may optionally be surface-modified include, without limitation, pigments that have been FDA approved. These may be suitable for cosmetic applications. Acceptable inorganic pigments that may be used in cosmetics are set forth in 21 C.F.R. §§ 70-82, which are hereby incorporated by reference in their entirety.

In an embodiment, the one or more inorganic pigments may be selected from colored metal oxides, metal hydroxides, metal oxide hydrates, silicates, metal sulfides, complex metal cyanides, metal sulfates, bronze pigments, colored mica- or mica-based pigments coated with at least one metal oxide and/or a metal oxychloride, or combinations thereof. Further, the one or more inorganic pigments may be selected from titanium dioxide, carbon black, black titanium oxide, yellow iron oxide, black iron oxide, red iron oxide, ultramarine blue, iron blue, chromium oxide, chromium hydroxide, carmine, and shikonin, or combinations thereof. In further embodiments, the inorganic pigment may be selected from activated carbon, talc, iron oxides, titanium dioxide, zinc oxide, silica, cerium oxides, zirconium oxides, aluminium oxides, calcium carbonate, barium sulfate, chromium oxides, manganese oxides, bismuth oxychloride, ultramarine, calcium sulfate, alkali magnesium silicates or mixtures thereof. Similarly, the one or more inorganic pigments may be selected from titanium dioxide, zinc oxide, iron oxides, chromium oxide, chromium hydroxide, ultramarine, ferric blue, mica, mica coated with titanium dioxide, mica coated with titanium dioxide and with metal oxides, bismuth oxide chloride, coated bismuth oxide chloride, metal powder in flake form of aluminum, brass, bronze, copper, silver, gold, and mixtures thereof.

Carbon black is an example of a useful inorganic pigment. More specific but nonlimiting examples of carbon black pigments include those manufactured by Mitsubishi Chemical Corporation, Japan (such as, e.g., CARBON BLACK No. 2300, No. 900, MCF88, No. 33, No. 40, No. 45, No. 52, MA7, MA8, MA100, and No. 2200B); various carbon black pigments of the RAVEN® series manufactured by Columbian Chemicals Company, Marietta, Ga., (such as, e.g., RAVEN® 5750, RAVEN® 5250, RAVEN® 5000, RAVEN® 3500, RAVEN® 1255, and RAVEN® 700); various carbon black pigments of the REGAL® series, BLACK PEARLS® series, the MOGUL® series, or the MONARCH® series manufactured by Cabot Corporation, Boston, Mass., (such as, e.g., REGAL® 400R, REGAL® 330R, REGAL® 660R, BLACK PEARLS® 700, BLACK PEARLS® 800, BLACK PEARLS® 880, BLACK PEARLS® 1100, BLACK PEARLS® 4350, BLACK PEARLS® 4750, MOGUL® E, MOGUL® L, and ELFTEX® 410); and various black pigments manufactured by Evonik Degussa Orion Corporation, Parsippany, N.J., (such as, e.g., Color Black FW1, Color Black FW2, Color Black FW2V, Color Black FW18, Color Black FW200, Color Black S150, Color Black S160, Color Black S170, PRINTEX® 35, PRINTEX® U, PRINTEX® V, PRINTEX® 140U, Special Black 5, Special Black 4A, and Special Black 4). An example of an organic black pigment includes aniline black, such as C.I. Pigment Black 1.

Nonlimiting examples of suitable inorganic pigments are commercially available under the trade names RONA®, COLORONA®, XIRONA®, DICHRONA® and TIMIRON® from Merck, ARIABEL® and UNIPURE® from Sensient, PRESTIGE® from ECKART COSMETIC COLORS AND SUNSHINE® from Sunstar. Useful color pigments with the trade name COLORONA® are, for example:
COLORONA COPPER, Merck, MICA, CI 77491 (IRON OXIDES),
COLORONA PASSION ORANGe, Merck, Mica, CI 77491 (Iron Oxides), Alumina,
COLORONA PATINA SILVER, Merck, MICA, CI 77499 (IRON OXIDES), CI 77891 (TITANIUM DIOXIDE),
COLORONA RY, Merck, CI 77891 (TITANIUM DIOXIDE), MICA, CI 75470 (CARMINE),
COLORONA ORIENTAL BEIGE, Merck, MICA, CI 77891 (TITANIUM DIOXIDE), CI 77491 (IRON OXIDES),
COLORONA DARK BLUE, Merck, MICA, TITANIUM DIOXIDE, FERRIC FERROCYANIDE,
COLORONA CHAMELEON, Merck, CI 77491 (IRON OXIDES), MICA,
COLORONA ABORIGINE AMBER, Merck, MICA, CI 77499 (IRON OXIDES), CI 77891 (TITANIUM DIOXIDE),
COLORONA BLACKSTAR BLUE, Merck, CI 77499 (IRON OXIDES), MICA,
COLORONA PATAGONIAN PURPLE, Merck, MICA, CI 77491 (IRON OXIDES), CI 77891 (TITANIUM DIOXIDE), CI 77510 (FERRIC FERROCYANIDE),
COLORONA RED BROWN, Merck, MICA, CI 77491 (IRON OXIDES), CI 77891 (TITANIUM DIOXIDE),
COLORONA RUSSET, Merck, CI 77491 (TITANIUM DIOXIDE), MICA, CI 77891 (IRON OXIDES),
COLORONA IMPERIAL RED, Merck, MICA, TITANIUM DIOXIDE (CI 77891), D&C RED NO. 30 (CI 73360),
COLORONA MAJESTIC GREEN, Merck, CI 77891 (TITANIUM DIOXIDE), MICA, CI 77288 (CHROMIUM OXIDE GREENS),
COLORONA LIGHT BLUE, Merck, MICA, TITANIUM DIOXIDE (CI 77891), FERRIC FERROCYANIDE (CI 77510),
COLORONA RED GOLD, Merck, MICA, CI 77891 (TITANIUM DIOXIDE), CI 77491 (IRON OXIDES),
COLORONA GOLD PLUS MP 25, Merck, MICA, TITANIUM DIOXIDE (CI 77891), IRON OXIDES (CI 77491),
COLORONA CARMINE RED, Merck, MICA, TITANIUM DIOXIDE, CARMINE,
COLORONA BLACKSTAR GREEN, Merck, MICA, CI 77499 (IRON OXIDES),
COLORONA BORDEAUX, Merck, MICA, CI 77491 (IRON OXIDES),
COLORONA BRONZE, Merck, MICA, CI 77491 (IRON OXIDES),
COLORONA BRONZE FINE, Merck, MICA, CI 77491 (IRON OXIDES),
COLORONA FINE GOLD MP 20, Merck, MICA, CI 77891 (TITANIUM DIOXIDE), CI 77491 (IRON OXIDES)
COLORONA SIENNA FINE, Merck, CI 77491 (IRON OXIDES), MICA
COLORONA SIENNA, Merck, MICA, CI 77491 (IRON OXIDES)
COLORONA PRECIOUS GOLD, Merck, Mica, CI 77891 (Titanium dioxide), Silica, CI 77491 (Iron oxides), Tin oxide,
COLORONA SUN GOLD SPARKLE MP 29, Merck, MICA, TITANIUM DIOXIDE, IRON OXIDES, MICA, CI 77891, CI 77491 (EU),
COLORONA MICA BLACK, Merck, CI 77499 (Iron oxides), Mica, CI 77891 (Titanium dioxide)
COLORONA BRIGHT GOLD, Merck, Mica, CI 77891 (Titanium dioxide), CI 77491 (Iron oxides)
COLORONA BLACKSTAR GOLD, Merck, MICA, CI 77499 (IRON OXIDES)

Other preferred color pigments with the trade name Xirona® are for example:
XIRONA GOLDEN SKY, Merck, Silica, CI 77891 (Titanium Dioxide), Tin Oxide
XIRONA CARIBBEAN BLUE, Merck, Mica, CI 77891 (Titanium Dioxide), Silica, Tin Oxide,
XIRONA KIWI ROse, Merck, Silica, CI 77891 (Titanium Dioxide), Tin Oxide,
XIRONA MAGIC MAUVE, Merck, Silica, CI 77891 (Titanium Dioxide), Tin Oxide.

In addition, preferred color pigments with the trade name UNIPURE® are for example:
UNIPURE RED LC 381 EM, Sensient CI 77491 (Iron Oxides), Silica
UNIPURE BLACK LC 989 EM, Sensient, CI 77499 (Iron Oxides), Silica
UNIPURE YELLOW LC 182 EM, Sensient, CI 77492 (Iron Oxides), Silica

The particle size (e.g., volume-weighted mean diameter (MV)) of the particles of inorganic pigments is not limited. In certain embodiments, the particle size of the inorganic pigments is less than 10 μm, less than 5 μm, less than 2 μm, less than 1 μm, less than 800 nm, less than 500 nm, or less than 300 nm. In various embodiments, the particle size of the inorganic pigments is from about 500 nm to about 10 μm, about 500 nm to about 5 μm, about 500 nm to about 2 μm, about 500 nm to about 1 μm, about 200 nm to about 1 μm, about 20 nm to about 800 nm. In further embodiments, the particle size in from about 30 nm to about 500 nm, about 50 nm to about 400 nm, or from about 100 nm to about 300 nm. Particle size variation in inorganic pigments leads to different hiding power, attributed to the light scattering effect. It results in smoother transitions between colors and a richer chromatic gradation of pigments.

Particle size can be determined by laser diffraction spectrometry, for example, using a Bettersizer® 2600, an LS™ Series Multi-Wavelength Particle Size Distributor from Beckman Coulter, Inc., or similar equipment. The laser diffraction method allows precise and efficient characterization of particle size by analyzing the scattering pattern of laser light.

The total amount of the one or more inorganic pigments, if present in the pigment dispersion, will vary. Nonetheless, in various embodiments, the pigment dispersion may include from about 5 to about 55 wt. % of one or more inorganic pigments, based on the total weight of the pigment dispersion. In further embodiments, the pigment dispersion may include from about 5 to about 50 wt. %, about 5 to about 45 wt. %, about 5 to about 40 wt. %, about 5 to about 35 wt. %, about 5 to about 30 wt. %, about 5 to about 25 wt. %, about 5 to about 20 wt. %, or about 5 to about 15 wt. %, based on the total weight of the pigment dispersion. In further embodiments, the pigment dispersion includes from about 10 to about 60 wt. %, about 10 to about 50 wt. %, about 10 to about 40 wt. %, about 10 to about 30 wt. %, or about 10 to about 30 wt. % of one or more inorganic pigments, based on the total weight of the pigment dispersion. In yet another embodiment, the pigment dispersion includes from about 20 to about 60 wt. %, about 20 to about 50 wt. %, about 20 to about 40 wt. %, or about 20 to about 30 wt. % of one or more inorganic pigments, based on the total weight of the pigment dispersion.

### Organic Pigments

Organic pigments are based on carbon chains and rings. They can be animal-based, vegetable-based, or synthetically manufactured. Like inorganic pigments, organic pigments are not soluble in water. However, some organic pigments are at least partially soluble in some strong solvents. Nonlimiting examples of organic pigments include nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

More specific but nonlimiting examples of organic pigments include carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof.

In a preferred embodiment, one or more organic pigments may be selected from carmine, quinacridone, phthalocyanine, Sorghum, blue pigments with the color index numbers CI 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with the color index numbers CI 61565, CI 61570, CI 74260, orange pigments with the color index numbers CI 11725, CI 15510, CI 45370, CI 71105, red pigments with the color index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, or mixtures thereof.

Nonlimiting examples of blue or cyan organic pigments include C.I. Pigment Blue 1, C.I. Pigment Blue 2, C.I. Pigment Blue 3, C.I. Pigment Blue 15, Pigment Blue 15:3, C.I. Pigment Blue 15:4, C.I. Pigment Blue 16, C.I. Pigment Blue 18, C.I. Pigment Blue 22, C.I. Pigment Blue 25, C.I. Pigment Blue 60, C.I. Pigment Blue 65, C.I. Pigment Blue 66, C.I. Vat Blue 4, C.I. Vat Blue 60, or combinations thereof.

Nonlimiting examples of magenta, red, or violet organic pigments include C.I. Pigment Red 1, C.I. Pigment Red 2, C.I. Pigment Red 3, C.I. Pigment Red 4, C.I. Pigment Red 5, C.I. Pigment Red 6, C.I. Pigment Red 7, C.I. Pigment Red 8, C.I. Pigment Red 9, C.I. Pigment Red 10, C.I. Pigment Red 11, C.I. Pigment Red 12, C.I. Pigment Red 14, C.I. Pigment Red 15, C.I. Pigment Red 16, C.I. Pigment Red 17, C.I. Pigment Red 18, C.I. Pigment Red 19, C.I. Pigment Red 21, C.I. Pigment Red 22, C.I. Pigment Red 23, C.I. Pigment Red 30, C.I. Pigment Red 31, C.I. Pigment Red 32, C.I. Pigment Red 37, C.I. Pigment Red 38, C.I. Pigment Red 40, C.I. Pigment Red 41, C.I. Pigment Red 42, C.I. Pigment Red 48 (Ca), C.I. Pigment Red 48 (Mn), C.I. Pigment Red 57 (Ca), C.I. Pigment Red 57:1, C.I. Pigment Red 88, C.I. Pigment Red 112, C.I. Pigment Red 114, C.I. Pigment Red 122, C.I. Pigment Red 123, C.I. Pigment Red 144, C.I. Pigment Red 146, C.I. Pigment Red 149, C.I. Pigment Red 150, C.I. Pigment Red 166, C.I. Pigment Red 168, C.I. Pigment Red 170, C.I. Pigment Red 171, C.I. Pigment Red 175, C.I. Pigment Red 176, C.I. Pigment Red 177, C.I. Pigment Red 178, C.I. Pigment Red 179, C.I. Pigment Red 184, C.I. Pigment Red 185, C.I. Pigment Red 187, C.I. Pigment Red 202, C.I. Pigment Red 209, C.I. Pigment Red 219, C.I. Pigment Red 224, C.I. Pigment Red 245, C.I. Pigment Red 286, C.I. Pigment Violet 19, C.I. Pigment Violet 23, C.I. Pigment Violet 32, C.I. Pigment Violet 33, C.I. Pigment Violet 36, C.I. Pigment Violet 38, C.I. Pigment Violet 43, C.I. Pigment Violet 50, or combinations thereof. Any quinacridone pigment or a co-crystal of quinacridone pigments may be used.

Nonlimiting examples of yellow organic pigments include C.I. Pigment Yellow 1, C.I. Pigment Yellow 2, C.I. Pigment Yellow 3, C.I. Pigment Yellow 4, C.I. Pigment Yellow 5, C.I. Pigment Yellow 6, C.I. Pigment Yellow 7, C.I. Pigment Yellow 10, C.I. Pigment Yellow 11, C.I. Pigment Yellow 12, C.I. Pigment Yellow 13, C.I. Pigment Yellow 14, C.I. Pigment Yellow 16, C.I. Pigment Yellow 17, C.I. Pigment Yellow 24, C.I. Pigment Yellow 34, C.I. Pigment Yellow 35, C.I. Pigment Yellow 37, C.I. Pigment Yellow 53, C.I. Pigment Yellow 55, C.I. Pigment Yellow 65, C.I. Pigment Yellow 73, C.I. Pigment Yellow 74, C.I. Pigment Yellow 75, C.I. Pigment Yellow 77, C.I. Pigment Yellow 81, C.I. Pigment Yellow 83, C.I. Pigment Yellow 93, C.I. Pigment Yellow 94, C.I. Pigment Yellow 95, C.I. Pigment Yellow 97, C.I. Pigment Yellow 98, C.I. Pigment Yellow 99, C.I. Pigment Yellow 108, C.I. Pigment Yellow 109, C.I. Pigment Yellow 110, C.I. Pigment Yellow 113, C.I. Pigment Yellow 114, C.I. Pigment Yellow 117, C.I. Pigment Yellow 120, C.I. Pigment Yellow 122, C.I. Pigment Yellow 124, C.I. Pigment Yellow 128, C.I. Pigment Yellow 129, C.I. Pigment Yellow 133, C.I. Pigment Yellow 138, C.I. Pigment Yellow 139, C.I. Pigment Yellow 147, C.I. Pigment Yellow 151, C.I. Pigment Yellow 153, C.I. Pigment Yellow 154, C.I. Pigment Yellow 155, C.I. Pigment Yellow 167, C.I. Pigment Yellow 172, C.I. Pigment Yellow 180, C.I. Pigment Yellow 185, and C.I. Pigment Yellow 213, or combinations thereof.

Nonlimiting examples of green organic pigments include C.I. Pigment Green 1, C.I. Pigment Green 2, C.I. Pigment Green 4, C.I. Pigment Green 7, C.I. Pigment Green 8, C.I. Pigment Green 10, C.I. Pigment Green 36, and C.I. Pigment Green 45.

Nonlimiting examples of brown organic pigments include C.I. Pigment Brown 1, C.I. Pigment Brown 5, C.I. Pigment Brown 22, C.I. Pigment Brown 23, C.I. Pigment Brown 25, C.I. Pigment Brown 41, and C.I. Pigment Brown 42, or combinations thereof.

Nonlimiting examples of orange organic pigments include C.I. Pigment Orange 1, C.I. Pigment Orange 2, C.I. Pigment Orange 5, C.I. Pigment Orange 7, C.I. Pigment Orange 13, C.I. Pigment Orange 15, C.I. Pigment Orange 16, C.I. Pigment Orange 17, C.I. Pigment Orange 19, C.I. Pigment Orange 24, C.I. Pigment Orange 34, C.I. Pigment Orange 36, C.I. Pigment Orange 38, C.I. Pigment Orange 40, C.I. Pigment Orange 43, C.I. Pigment Orange 66, or combinations thereof.

In certain embodiments, the dispersion may optionally include one or more organic pigment selected from carmine, quinacridone, phthalocyanine, sorghum, blue pigments with the color index numbers CI 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, or combinations thereof.

The particle size (e.g., volume-weighted mean diameter (MV)) of the particles of organic pigments is not limited. In certain embodiments, the particle size of the organic pigments is less than 15 μm, less than 10 μm, less than 5 μm, less than 2 μm, less than 1 μm, less than 800 nm, less than 500 nm, or less than 300 nm. In various embodiments, the particle size of the organic pigments is from about 500 nm to about 15 μm, about 500 nm to about 10 μm, about 1 μm to about 10 μm, about 2 μm to about 10 μm, about 5 μm to about 15 μm, about 5 μm to about 10 μm. In certain embodiments, it can be preferably to include smaller particles of organic pigments, for example, from about 200 nm to about 1 μm, about 20 nm to about 800 nm. In further embodiments, the particle size in from about 30 nm to about 500 nm, about 50 nm to about 400 nm, or from about 100 nm to about 300 nm.

Particle size can be determined by laser diffraction spectrometry, for example, using a Bettersizer® 2600, an LS™ Series Multi-Wavelength Particle Size Distributor from Beckman Coulter, Inc., or similar equipment. The laser diffraction method allows precise and efficient characterization of particle size by analyzing the scattering pattern of laser light.

The total amount of the one or more organic pigments, if present in the pigment dispersion, will vary. Nonetheless, in various embodiments, the pigment dispersion can include from about 5 to about 55 wt. % of one or more organic pigments, based on the total weight of the pigment dispersion. In further embodiments, the pigment dispersion may include from about 5 to about 50 wt. %, about 5 to about 45 wt. %, about 5 to about 40 wt. %, about 5 to about 35 wt. %, about 5 to about 30 wt. %, about 5 to about 25 wt. %, about 5 to about 20 wt. %, or about 5 to about 15 wt. % of one or more organic pigments, based on the total weight of the pigment dispersion. In further embodiments, the pigment dispersion includes from about 10 to about 60 wt. %, about 10 to about 50 wt. %, about 10 to about 40 wt. %, about 10 to about 30 wt. %, or about 10 to about 30 wt. % of one or more organic pigments, based on the total weight of the pigment dispersion. In yet another embodiment, the pigment dispersion includes from about 20 to about 60 wt. %, about 20 to about 50 wt. %, about 20 to about 40 wt. %, or about 20 to about 30 wt. % of one or more organic pigments, based on the total weight of the pigment dispersion.

### Solid Fatty Compounds

The pigment dispersions may optionally include one or more solid fatty compounds. A solid fatty compound has a melting point above 25° C. at atmospheric pressure, i.e., they are solid at 25° C. and atmospheric pressure. Although the pigment dispersions may include one or more fatty compounds, the one or more fatty compounds are typically not solid when incorporated into the pigment dispersion. The one or more solid fatty compounds is usually dissolved by the other components of the pigment dispersion, for example, by the one or more solvents, preferably one or more oils, capable of dissolving the hydrophobic polymer of (a).

Nonlimiting examples of solid fatty compounds include solid animal and solid vegetable oils and fats, fully hydrogenated or partially hydrogenated vegetable and animal oils and fats, saturated fatty acids, partially hydrogenated or fully hydrogenated fatty acids, fatty acid esters, saturated, partially hydrogenated or fully hydrogenated monoglycerides, diglycerides and triglycerides, phospholipids, lecithins, partially hydrogenated or completely hydrogenated phospholipids and lecithins, lysolecithins and lysophosphatidylcholine, animal waxes, plant waxes, mineral waxes, synthetic waxes, wax esters, saturated and unsaturated fatty alcohols, fatty alcohol ethers/esters; solid saturated and unsaturated hydrocarbons (paraffins); solid silicones and silicone ethers/esters; polyol ethers/esters: glycerol ethers/esters, sorbitan, sorbitan stearate, glyceryl ricinoleate; polyglycerols and their ethers/esters, hydrophobic gelling agents: silicon dioxide, polyethylenes, or mixtures thereof.

In various embodiments, one or more of the solid fatty compounds is a wax, for example, a vegetable wax, an animal wax, a mineral, a synthetic wax and/or petroleum derived wax. Nonlimiting examples include Bayberry wax, candelilla wax, carnauba wax, castor wax, esparto wax, japan wax, ouricury wax, rice bran wax, soy wax, tallow tree wax, beeswax, Chinese wax, lanolin wax (wool wax), shellac wax, spermaceti wax, and mixtures thereof, paraffin wax, microcrystalline wax, ceresin wax, montan wax, ozocerite wax, polyethylene wax, peat wax.

In further embodiments, one or more of the solid fatty compounds may be a resin, for example, plant and/or animal and/or petroleum derived resin and/or a synthetic resin. Nonlimiting examples include asphaltite, Utah resin (petroleum bitumens), shellac, copals, dammars, mastic, sandarac, frankincense, elemi, turpentine, copaiba, gum resins, Aleppo pine resin, bisphenol A diglycidyl ether and silicone resins.

Solid fatty alcohols include those represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include cetyl alcohol, stearyl alcohol, behenyl alcohol and mixtures thereof, preferably cetyl alcohol, behenyl alcohol, cetearyl alcohol, and mixtures thereof.

Nonlimiting examples of solid fatty acids include decanoic acid, lauric acid, myristic acid, palmitic acid, stearic acid, arachidic acid or behenic acid. Solid fatty alcohols may be linear, unsaturated 1-alkanols with at least 12 carbon atoms. Examples of solid fatty alcohols are lauryl alcohol, myristyl alcohol, cetyl alcohol, stearyl alcohol, arachidyl alcohol, behenyl alcohol, or mixtures thereof.

In certain embodiments, the one or more solid fatty compounds are selected from waxes, fatty alcohols, solid fatty acids, or combinations thereof. Nonlimiting examples include candelilla wax, carnauba wax, castor wax, beeswax, lanolin Wax, ozokerite wax, microcrystalline wax, sunflower wax, tribehenin, cetyl alcohol, stearyl alcohol, cetearyl alcohol, lanolin alcohol, or mixtures thereof.

The total amount of the one or more solid fatty compounds in the pigment dispersions, if present, will vary. Nonetheless, the pigment dispersions may optionally include from about 0.01 to about 20 wt. % of one or more solid fatty compounds, based on the total weight of the pigment dispersion. In further embodiments, the pigment dispersions include from about 0.01 to about 15 wt. %, about 0.01 to about 10 wt. %, about 0.01 to about 5 wt. %, about 0.01 to about 3 wt. %, of one or more solid fatty compounds, based on the total weight of the pigment dispersions. In other embodiments, the pigment dispersions include from about 0.1 to about 20 wt. %, about 0.1 to about 15 wt. %, about 0.1 to about 10 wt. %, about 0.1 to about 5 wt. %, about 1 to about 20 wt. %, about 1 to about 15 wt. %, about 1 to about 10 wt. %, about 1 to about 5 wt. %, about 2 to about 20 wt. %, about 2 to about 15 wt. %, about 2 to about 10 wt. %, about 2 to about 5 wt. %, about 5 to about 20 wt. %, about 5 to about 15 wt. %, or about 5 to about 10 wt. % of one or more solid fatty compounds, based on the total weight of the pigment dispersion.

### Oil Thickening Agents

The pigment dispersions may optionally include one or more oil thickening agents. An “oil thickening agent” means a material which when combined with pigment dispersion results in a thickening of the pigment dispersion. Nonlimiting examples of oil thickening agents include polymers, gums, organoclays, polyethylenes, silica, or combinations thereof. More specific but nonlimiting examples include acrylate copolymer, hectorite gel, guar gum, cellulose gum, Caesalpinia spinosa gum, gum Arabic, starch, silica, silica dimethyl silylate, behenate, glyceryl dibehenate, behenyl behenate, or mixtures thereof. In various embodiments, polyalcohol acrylates may be useful, for examples, poly C10-30 alkyl acrylate. The oil thickening agents can be selected from semi-crystalline or crystalline polymers and/or semi-crystalline or crystalline waxes.

In one embodiment, one or more oil thickening agents are selected from polymeric thickening agents and inorganic thickening agents. In a further embodiment, at least one polymeric thickening agent can be an amorphous polymer formed by polymerization of an olefin. In a further embodiment, the olefin can be an ethylenically unsaturated elastomer monomer. Examples of olefins include, but are not limited to, ethylenic carbide monomers, for example those comprising one or two ethylenic unsaturations, comprising from 2 to 5 carbon atoms, such as ethylene, propylene, butadiene, or isoprene.

In various embodiments, the oil thickening agents may be polycondensation products of the polyamide type resulting from condensation between (α) at least one acid chosen from dicarboxylic acids comprising at least 32 carbon atoms, such as the dimeric fatty acids and (β) alkylenediamines, such as ethylenediamine, wherein the polymeric polyamide comprises at least one terminal carboxylic acid group esterified or amidated with at least one monoalcohol or one monoamine comprising from 12 to 30 linear and saturated carbon atoms, for example ethylenediamine/stearyl dilinoleate copolymers such as that marketed under the name Uniclear 100 VG® by Arizona Chemical.

The oil thickening agent may be selected from inorganic thickening agents of oils, such as organophilic clay or at least one pyrogenic silica. As used herein, “organophilic clays” mean clays modified with chemical compounds rendering the clay capable of swelling in oily media. The clays are products already well known in themselves, which are for example described in the book “Mineralogy of clays, S. Caillère, S. Hénin, M. Rautureau, 2nd edition 1982, Masson”, the teaching of which is incorporated herein by reference in its entirety. In one embodiment, the at least one clay is a silicate comprising a cation which may be chosen from the cations of calcium, magnesium, aluminium, sodium, potassium, and lithium. Examples of such clays include, but are not limited to, the clays of the smectite family such as montmorillonites, hectorites, bentonites, beidellites, saponites, and the vermiculite family, stevensite, and chlorites. These clays can be of natural or synthetic origin.

The total amount of the one or more oil thickening agents, if present, will vary. Nonetheless, in certain embodiments, the pigment dispersions may include from about 0.1 to about 10 wt. % of one or more oil thickening agents, based on the total weight of the pigment dispersion. In further embodiments, the pigment dispersions may have from about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. % of one or more oil thickening agents, based on the total weight of the pigment dispersion.

### Lipophilic Film-Forming Polymers

The term “film-forming polymer” refers to a polymer able to form alone or in the presence of an auxiliary film-forming agent, a macroscopically continuous film that adheres to the keratin fibers, and preferably a cohesive film. Among the film-forming polymers, mention can be made of radical or polycondensate type synthetic polymers, polymers of natural origin, and mixtures thereof.

Nonlimiting examples of lipophilic film-forming polymers include polyalkylenes, and in particular C₂-C₂₀ alkene copolymers, such as polybutene, alkylcelluloses with a linear or branched, saturated or unsaturated C₁ to C₈ alkyl radical, such as ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymers.

In various embodiments, the pigment dispersion may include one or more lipophilic film-forming polymer chosen from (i) silicone resins of MQ type, (ii) silsesquioxane resins, (iii) vinyl polymers grafted with a carbosiloxane dendrimer, (iv) polyalkylsiloxanes and polyarylsiloxanes, or (v) mixtures thereof.

Furthermore, in some instances, the lipophilic film-forming polymer may be selected from vinyl ester polymers and copolymers, vinylpyrrolidone copolymers, or mixtures thereof.

The total amount of the one or more lipophilic film-forming polymers, if present, will vary. Nonetheless, in certain embodiments, the pigment dispersions may include from about 0.1 to about 10 wt. % of one or more lipophilic film-forming polymers, based on the total weight of the pigment dispersion. In further embodiments, the pigment dispersions may have from about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. % of one or more lipophilic film-forming polymers, based on the total weight of the pigment dispersion.

### Miscellaneous Ingredients

The pigment dispersions optionally include one or more miscellaneous ingredients. Miscellaneous ingredients are ingredients that are compatible with the pigment dispersions and do not disrupt or materially affect the basic and novel properties of the pigment dispersions. Nonlimiting examples of ingredients include preservatives, fragrances, pH adjusters, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the pigment dispersions, if present, will vary. Nonetheless, the pigment dispersions may include from about 0.1 to about 15 wt. % of the one or more miscellaneous ingredients, based on the total weight of the pigment dispersion. In further embodiments, the pigment dispersions include from about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of one or more miscellaneous ingredients, based on the total weight of the pigment dispersion.

### Oil-In-Water Emulsions

As noted above, the pigment dispersions may be used to generate oil-in-water emulsions. A variety of cosmetic and personal care products are preferably formulated as oil-in-water emulsions. The pigment dispersions can serve as the oil phase of such oil-in-water emulsions. The amount of oil phase (the pigment dispersion) and the amount of an aqueous phase can vary depending on the required properties and form of the oil-in-water emulsion and how it should function. Nonetheless, the oil phase (the pigment dispersion) and the aqueous phase are typically in a weight ratio of from about 1:5 to about 5:1 (oil phase: aqueous phase). More preferably, however, the weight ratio of the oil phase and the aqueous phase is from about 1:5 to about 1:2, about 1:5 to about 1:1, about 1:3 to about 1:1, or about 1:2 to about 1:1 (oil phase: aqueous phase).

More specifically, the oil phase (pigment dispersion) may constitute about 5 to about 65 wt. % of an oil-in-water emulsion. In further embodiments, the oil phase constitutes from about 5 to about 60 wt. %, about 5 to about 50 wt. %, about 5 to about 45 wt. %, about 5 to about 40 wt. %, about 5 to about 35 wt. %, about 5 to about 25 wt. %, about 5 to about 20 wt. %, about 10 to about 65 wt. %, about 10 to about 60 wt. %, about 10 to about 50 wt. %, about 10 to about 40 wt. %, about 10 to about 30 wt. %, about 10 to about 25 wt. %, about 25 to about 60 wt. %, about 25 to about 50 wt. %, about 25 to about 40 wt. %, about 30 to about 65 wt. %, about 30 to about 60 wt. %, or about 30 to about 50 wt. %, based on the total weight of the oil-in-water emulsion.

The aqueous phase may constitute about 40 to about 95 wt. % of the oil-in-water emulsion. In further embodiments, the aqueous phase constitutes from about 40 to about 90 wt. %, about 40 to about 80 wt. %, about 40 to about 70 wt. %, about 50 to about 95 wt. %, about 50 to about 90 wt. %, about 50 to about 80 wt. %, about 50 to about 70 wt. %, about 60 to about 95 wt. %, about 60 to about 90 wt. %, or about 60 to about 80 wt. % of the oil-in-water emulsion.

### Water

Water is typically a primary component of the aqueous phase. Therefore, the aqueous phase can include high amounts of water, for example, from about 50 to about 100 wt. %, based on the total weight of the aqueous phase. In various embodiments, however, the aqueous phase includes from about 50 to about 99 wt. %, about 50 to about 95 wt. %, about 50 to about 90 wt. %, about 50 to about 80 wt. %, about 50 to about 70 wt. %, about 60 to about 99 wt. %, about 60 to about 95 wt. %, about 60 to about 90 wt. %, about 60 to about 80 wt. %, about 70 to about 99 wt. %, about 70 to about 95 wt. %, about 70 to about 90 wt. %, about 70 to about 80 wt. % of water.

### Water-Soluble Solvents

The aqueous phase may optionally include one or more water soluble solvents. The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, mono-alcohols (for example C₂₋₈, or C₂₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example C₂₋₆ or C₂₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof. In a preferred embodiment, the composition include one or more glycols selected from propylene glycol, butylene glycol, pentylene glycol, hexylene glycol, caprylyl glycol, dipropylene glycol, and mixtures thereof.

The total amount of the one or more water soluble solvents in the aqueous phase, if present, will vary. Nonetheless, the aqueous phase may include from about 0.1 to about 30 wt. % of one or more water soluble solvents, based on the total weight of the aqueous phase. In further embodiments, the aqueous phase includes from about 0.1 to about 20 wt. %, about 0.1 to about 15 wt. %, about 0.1 to about 10 wt. %, about 0.1 to about 5 wt. %, about 1 to about 30 wt. %, about 1 to about 30 wt. %, about 1 to about 15 wt. %, about 1 to about 10 wt. %, about 1 to about 5 wt. %, about 2 to about 30 wt. %, about 2 to about 20 wt. %, about 2 to about 15 wt. %, about 2 to about 10 wt. %, or about 2 to about 8 wt. % of one or more water soluble solvents, based on the total weight of the aqueous phase.

### Water-Soluble Dyes

The aqueous phase may include one or more water-soluble dyes. As the name suggests, water-soluble dyes are dyes that are solubilized by the aqueous phase. Nonlimiting examples of water-soluble dyes include Red No. 2 (Acid Red 27: C. I. 16185), Red No. 3 (Acid Red 51: C. I. 45430), Red No. 102 (Acid Red 18: C. I. 16255), Red No. 104 (1) (Acid Red 92: C.I. 45410), Red No. 105 (1) (Acid Red 94: C.I. 45440), Red No. 106 (Acid Red 52: C.I. 45100), Red No. 227 (Acid Red 33: C.I. 17200), Red No. 230 (1) and Red No. 230 (2) (both Acid Red 87: C.I. 45380), Red No. 231 (Acid Red 92: C.I. 45410), Red No. 232 (Acid Red 94: C.I. 45440), Yellow No. 4 (Acid Yellow 23: C.I. 19140), Yellow No. 5 (Food Yellow 3: C.I. 15985), Yellow No. 202 (1) and Yellow No. 202 (2) (both Acid Yellow 73: C.I. 45350), Yellow No. 203 (Acid Yellow 3: C.I. 47005), Green No. 3 (Food Green: C.I. 42053), Green No. 201 (Acid Green 25: C.I. 61570), Green No. 204 (Solvent Green 7: C.I. 59040), Green No. 205 (Acid Green 5: C.I. 42095), Blue No. 1 (Food Blue 2: C.I. 42090), Blue No. 2 (Acid Blue 74: C.I. 73015), Blue No. 202 (Acid Blue 5: C.I. 42052), Blue No. 205 (Acid Blue 9: C.I. 42090), Orange No. 205 (Acid Orange 7: C.I. 15510), Orange No. 207 (Acid Red 95: C.I. 45425), and Brown No. 201 (Acid Orange 24: C.I. 20170); Red No. 201 (Pigment Red 57-1: C.I. 15850), Red No. 202 (Pigment Red 57: C.I. 15850), Red No. 203 (Pigment Red 53: C.I. 15585), Red No. 204 (Pigment Red 53 (Ba): C.I. 15585), Red No. 205 (Pigment Red 49 (Na): C.I. 15630), Red No. 206 (Pigment Red 49 (Ca): C.I. 15630), Red No. 207 (Pigment Red 49 (Ba): C.I. 15630), Red No. 208 (Pigment Red 49 (Sr): C.I. 15630), Red No. 215 (Solvent Red 49: C.I. 45170), Red No. 218 (Solvent Red 48: C.I. 45410), Red No. 219 (Pigment Red 64: C.I. 15800), Red No. 220 (Pigment Red 63 (Ca): C.I. 15880), Red No. 221 (Pigment Red 3: C.I. 12120), Red No. 223 (Solvent Red 43: C.I. 45380), Red No. 225 (Solvent Red 23: C.I. 26100), Red No. 226 (Vat Red 1: C.I. 73360), Yellow No. 201 (Acid Yellow 73: C.I. 45350), Yellow No. 204 (Solvent Yellow 33: C.I. 47000), Yellow No. 205 (Pigment Yellow 12: C.I. 21090), Green No. 202 (Solvent Green 3: C.I. 61565), Blue No. 201 (Vat Blue 1: C.I. 73000), Blue No. 204 (Vat Blue 6: C.I. 69825), Blue No. 404 (Pigment Blue 15: C.I. 74160), Orange No. 201 (Solvent Red 72: C.I. 45370), Orange No. 203 (Pigment Orange 5: C.I. 12075), Orange No. 204 (Pigment Orange 13: C.I. 21110), Orange No. 206 (Solvent Red 73: C.I. 45425), Orange No. 401 (Pigment Orange 1: C.I. 11725), Orange No. 402 (Acid Orange 20: C.I. 14600), Orange No. 403 (Solvent Orange No. 2: C.I. 12100, Black No. 401 (Acid Black 1: C.I. 20470), and Violet 201 (Solvent Violet 13: C.I. 60725), or mixtures thereof.

The total amount of the water-soluble dyes will vary but can be from about 0.01 to about 10 wt. %, based on the total weight of the aqueous phase. In further embodiments, the aqueous phase includes from about 0.1 to about 10 wt. %, about 0.1 to about 8 wt. %, about 0.1 to about 5 wt. %, about 0.1 to about 3 wt. %, or about 0.1 to about 2 wt. %, based on the total weight of the aqueous phase.

### Surfactants

Surfactants are commonly used in the manufacture of emulsions and may be preferably included in the oil-in-water emulsions of the instant disclosure. For purposes of the instant disclosure, the term “surfactant” includes emulsifiers and detergents. Surfactants, or surface-active agents, are compounds that lower the surface tension between two liquids or between a liquid and a solid. Surfactants are amphiphilic, meaning that they contain hydrophilic (water-loving) head groups and hydrophobic (water-hating, or oil-loving) tails. Surfactants adsorb at the interface between oil and water, thereby decreasing the surface tension.

For purposes of the instant disclosure, an “emulsifier” is a surfactant that stabilizes emulsions. Emulsifiers coat droplets within an emulsion and prevent them from coming together, or coalescing. An “emulsion” is a mixture of two or more liquids, with or without an emulsifier, that are normally immiscible. One of the liquids, the “dispersed phase,” forms droplets in the other liquid, the “continuous phase.”

A “detergent” is a surfactant that has cleaning properties in dilute solutions and is typically anionic.

The surfactants can be anionic, cationic, amphoteric (zwitterionic), or nonionic. Preferably, the emulsions of the instant case include one or more surfactants selected from anionic surfactants, amphoteric (zwitterionic) surfactants, nonionic surfactants, or mixtures thereof. In various embodiments, the emulsions are preferably free or essentially free from cationic surfactants. In other embodiments, the emulsions include one or more cationic surfactants. In preferred embodiments, the emulsions contain one or more biosurfactants, one or more anionic surfactants, optionally, one or more nonionic surfactants, or mixtures thereof.

In a preferred embodiment, the emulsions of the instant disclosure include a plurality of surfactants, wherein the plurality of surfactants include one or more biosurfactants and one or more surfactants other than the one or more biosurfactants. In further embodiments, the emulsions of the instant disclosure include one or more biosurfactants, one or more anionic surfactants, and optionally, one or more nonionic surfactants. In yet further embodiments, the emulsions include one or more cationic surfactants.

The total amount of the one or more surfactants will vary but may be from about 0.5 to about 20 wt. %, based on the total amount of the aqueous phase. In further embodiments, the total amount of the one or more surfactants in the aqueous phase is from about 0.5 to about 15 wt. %, about 0.5 to about 12 wt. %, about 0.5 to about 8 wt. %, about 0.5 to about 6 wt. %, about 1 to about 20 wt. %, about 1 to about 15 wt. %, about 1 to about 12 wt. %, about 1 to about 10 wt. %, about 1 to about 8 wt. %, about 1 to about 6 wt. %, about 2 to about 20 wt. %, about 2 to about 15 wt. %, about 2 to about 12 wt. %, about 2 to about 10 wt. %, about 2 to about 8 wt. %, about 2 to about 6 wt. %, about 3 to about 20 wt. %, about 3 to about 15 wt. %, about 3 to about 12 wt. %, about 3 to about 10 wt. %, about 3 to about 8 wt. %, about 3 to about 6 wt. %, about 4 to about 20 wt. %, about 4 to about 15 wt. %, about 4 to about 12 wt. %, about 4 to about 10 wt. %, about 4 to about 8 wt. %, or about 4 to about 6 wt. %, based on the total weight of the aqueous phase.

### Biosurfactant

The emulsions of the instant disclosure may optionally include one or more biosurfactants. Biosurfactants are amphiphilic molecules, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

Biosurfactants are environmentally friendly, biodegradable, and non-toxic and may be classified into high and low molecular weight biosurfactants. Low molecular weight biosurfactant efficiently lower surface and interfacial tension, and high molecular weight biosurfactants are more effective as emulsion-stabilizing agents. Examples of low molecular weight biosurfactants include glycolipids, such as rhamnolipids, sophorolipids, lipopeptidesm, and trehalolipids. These low molecular weight biosurfactants have hydrophilic heads comprised of sugar units linked glycosidically with hydrophobic non-polar parts. Examples of high molecular weight biosurfactants include polysaccharides, lipopolysaccharides, proteins, and lipoproteins. Polysaccharide-based biosurfactant can be classified into sorbitan esters, sucrose esters and glucose-based surfactants that include alkyl polyglycosides and fatty acid glucamides.

Nonlimiting examples of biosurfactants include liptopeptides such as surfactin; fatty acids and phospholipids, polymeric matrix biosurfactants; particulate biosurfactants; and bacterial biosurfactants composed of polysaccharides, proteins, lipopolysaccharides, lipoproteins or complex mixtures of these biopolymers.

Nonlimiting examples of commercially available biosurfactants include alkyl polyglycoside available under the trademark ECOSENSE® 3000 from Dow Chemical®; D-glucopyranose, oligomeric, decyl octyl glycosides available under the trademark GLUCOPON® 215 from BASF Corporation®; rhamnolipids available under the trademark REWOFERM® SL ONE from Evonik®; D-Glucitol, 1-deoxy-1-(methylamino)-, N-coco acyl derivatives available under the trademark GLUCOTAIN® from Clariant®; rhamnolipids from Jeneil Biotech®, and BioLoop® surfactants from Lankem® Ltd.

In one embodiment, the microbial biosurfactant is a glycolipid such as rhamnolipids (RLP), sophorolipids (SLP), trehalose lipid or mannosylerythritol lipid (MEL). The biosurfactants can be added in purified form or can be present in the microbe-based composition as a result of microbial growth. The biosurfactant may be a sophorolipid. In some embodiments, the biosurfactant can also be a lipopeptide, such as surfactin, and/or a rhamnolipid.

In some embodiments, a blend of biosurfactants is present. Preferably the blend comprises a rhamnolipid, and optionally one or both of a mannosylerythritol lipid, a surfactin or a sophorolipid. In a preferred embodiment, the microbe is a non-pathogenic strain of Pseudomonas. Preferably, the strain is a producer of rhamnolipid (RLP) biosurfactants.

Other microbial strains including, for example, other fungal strains capable of accumulating significant amounts of, for example, glycolipid-biosurfactants can be used in accordance with the subject invention. Biosurfactants useful according to the present invention include mannoprotein, beta-glucan and other metabolites that have bio-emulsifying and surface/interfacial tension-reducing properties.

In various embodiments, the one or more biosurfactants are selected from surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof. In one embodiment, the biosurfactants are selected from one or more glycolipids such as, for example, rhamnolipids, rhamnose-d-phospholipids, trehalose lipids, trehalose dimycolates, trehalose monomycolates, mannosylerythritol lipids, cellobiose lipids, ustilagic acid and/or sophorolipids.

In various embodiments, the biosurfactant has an anionic character, for example, sophorolipids, trehalolipid and rhamnolipids. Preferable are the mono-rhamnolipids and di-rhamnolipids. The preferred alkyl chain length is from C8 to C12. The alkyl chain may be saturated or unsaturated.

The term “rhamnolipids” includes compounds of the general formula (II) and salts thereof,

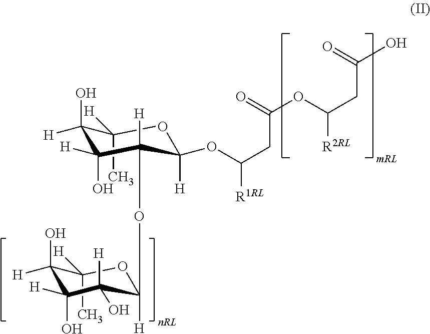

wherein,
mRL=2, 1 or 0,
nRL=1 or 0,
R^{1RL} and R^{2RL}=are independently organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CHs where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. 1-3-{1-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (II) or salts thereof, where nRL=1.

The term “mono-rhamnolipid” in the context of the present invention is understood to mean compounds of the general formula (II) or salts thereof, where nRL=0.

Distinct rhamnolipids are abbreviated according to the following nomenclature: “diRL-CXCY” are understood to mean di-rhamnolipids of the general formula (II), in which one of the residues R^{1RL} and R^{2RL}=(CH2)ₒ-CH₃ where o=X−4 and the remaining residue R¹ or R²=(CH₂)ₒ—CH₃ where o=Y−4.

“monoRL-CXCY” are understood to mean mono-rhamnolipids of the general formula (II), in which one of the residues R^{1RL} and R^{2RL}=(CH.sub.₂).sub.ₒ-CH.sub.₃ where o=X−4 and the remaining residue R^{1RL} or R^{2RL}=(CH₂)ₒ—CH₃ where o=Y−4. The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “: Z”, this signifies that the respective residue R^{1RL} and/or R^{2RL} is equal to an unbranched, unsubstituted hydrocarbon residue having X−3 or Y−3 carbon atoms having Z double bonds.

Methods for preparing the relevant rhamnolipids are disclosed, for example, in EP2786743 and EP2787065, which are incorporated herein by reference in their entirety. Rhamolipids can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been Improved for higher rhamnolipid titres by genetical modification can also be used in the context of the instant invention; such cells have for example been disclosed by Lei et al. in BIOTECHNOL LETT. 2020 June; 42 (6): 997-1002, which is incorporated herein by reference in its entirety. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789, which are incorporated herein by reference in their entirety.

Rhamnolipids produced by Pseudomonas aeruginosa are commercially available from Jeneil Biotech Inc., e.g. under the tradename ZONIX®, from Logos Technologies (technology acquired by Stepan), e.g. under the tradename NATSURFACT®, from Biotensidon GmbH, e.g. under the tradename RHAPYNAL®, from AGAE® technologies, e.g. under the name R90, R95, R95Md, R95Dd, from Locus Bio-Energy Solutions and from Shanghai Yusheng Industry Co. Ltd., e.g. under the tradename BIO-201 GLYCOLIPIDS®.

The total amount of the one or more biosurfactants in the emulsions, if present, will vary but is typically from about 0.1 to about 20 wt. %, based on the total weight of the aqueous phase. In further embodiments, the total amount of the one or more biosurfactants in the emulsions is from about 0.1 to about 15 wt. %, about 0.1 to about 10 wt. %, or about 0.1 to about 5 wt. %, based on the total weight of the aqueous phase. In a further embodiment, the total amount of the one or more biosurfactants is from about 0.5 to about 20 wt. %, about 0.5 to about 15 wt. %, about 0.5 to about 10 wt. %, or about 0.5 to about 5 wt. %, based on the total weight of the aqueous phase. In yet a further embodiment, the total amount of the one or more biosurfactants is from about 1 to about 20 wt. %, about 1 to about 15 wt. %, about 1 to about 10 wt. %, or about 1 to about 5 wt. %, based on the total weight of the aqueous phase. In a preferred embodiment, the total amount of the one or more biosurfactants is from about 2 to about 20 wt. %, about 2 to about 15 wt. %, about 2 to about 10 wt. %, about 2 to about 5 wt. %, about 3 to about 20 wt. %, about 3 to about 15 wt. %, about 3 to about 10 wt. %, about 3 to about 5 wt. %, about 2 to about 8 wt. %, about 2 to about 6 wt. %, about 3 to about 8 wt. %, or about 3 to about 6 wt. %, based on the total weight of the aqueous phase.

### Anionic Surfactants

In various embodiment, the emulsions of the instant disclosure include one or more anionic surfactants. Common popular anionic surfactants include sodium lauryl sulfate and sodium laureth ether sulfate, which may be included. The one or more anionic surfactants, if present, may also be non-sulfate anionic surfactants. Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. In some cases, however, acyl taurates are preferred and therefore the one or more non-sulfate anionic surfactants include at least one acyl taurate. In other cases, acyl isethionates are preferred and therefore the one or more non-sulfate anionic surfactants include at least one acyl isethionate.

In yet other cases, a combination of acyl taurates and acyl isethionates may be used. Thus, the emulsions may include two or more non-sulfate anionic surfactants comprising anionic surfactants selected from acyl taurates, acyl isethionates, or combinations thereof.

The total amount of the one or more anionic surfactants in the emulsions of the instant disclosure, if present, will vary, but may be in an amount from about 0.01 to about 10 wt. %, based on the total weight of the aqueous phase. In further embodiments, the emulsions includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more anionic surfactants, based on the total weight of the aqueous phase.

### Amphoteric Surfactants

The emulsions of the instant disclosure may optionally include one or more amphoteric surfactants. Nonlimiting examples of amphoteric surfactants include betaines, alkyl amphoacetates and alkyl amphodiacetates, alkyl sulltaines, alkyl amphopropionates, and combinations thereof.

The total amount of the one or more amphoteric surfactants in the emulsions may be from about 0.01 to about 10 wt. %, based on the total weight of the aqueous phase. In further embodiments, the emulsions include from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more amphoteric surfactants, based on the total weight of the aqueous phase.

### Nonionic Surfactants

In various embodiments, the emulsions include one or more nonionic surfactants. In various embodiments, at least one of the surfactants in the emulsions is a nonionic surfactant, especially a nonionic surfactants that provides good emulsifying properties. Nonlimiting examples of useful nonionic surfactants include alkoxylated fatty alcohols, alkoxylated polyol esters, alkoxylated glycerides, glucosides, alkanolamides, sorbitan derivatives, or combinations thereof.

The total amount of the one or more nonionic surfactants in the emulsions may be from about 0.01 to about 10 wt. %, based on the total weight of the aqueous phase.

In further embodiments, the emulsions includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more nonionic surfactants, based on the total weight of the aqueous phase.

### Cationic Surfactants

The emulsions may optionally include one or more cationic surfactants. The term “cationic surfactant” as used in the present disclosure is a surfactant that may be positively charged when it is contained in the emulsions according to the present disclosure. The cationic surfactant may bear one or more positive permanent charges or may contain one or more functional groups that are cationizable in the compositions.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

The one or more cationic surfactants may be selected from quaternary ammonium compounds, fatty dialkylamines, or mixtures thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and combinations thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, and combinations thereof.

In various embodiments, the one or more cationic surfactants are preferably selected from cetrimonium chloride, behentrimonium chloride, behentrimonium methosulfate, stearamidopropyl dimethylamine, brassicamidopropyl dimethylamine or a mixture thereof.

The total amount of the one or more cationic surfactants, if present, will vary but may be in an amount from about 0.01 to about 10 wt. %, based on the total weight of the aqueous phase. In further embodiments, the emulsions include from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more cationic surfactants, based on the total weight of the aqueous phase.

### Miscellaneous Ingredients

The anhydrous phase optionally includes one or more miscellaneous ingredients. Miscellaneous ingredients are ingredients that are compatible with the aqueous phase (and the emulsion) and do not disrupt or materially affect the basic and novel properties of the aqueous phase (and the emulsion). Nonlimiting examples of miscellaneous ingredients include preservatives, fragrances, pH adjusters, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the aqueous phase, if present, will vary. Nonetheless, the aqueous phase may include from about 0.1 to about 15 wt. % of the one or more miscellaneous ingredients, based on the total weight of the second aqueous phase. In further embodiments, the aqueous phase includes from about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of one or more miscellaneous ingredients, based on the total weight of the aqueous phase.

### Droplet Size

In various embodiments, the average droplet size of the oil phase in the emulsions forming the emulsions is from about 10 nm to about 2 μm, about 10 nm to about 1.5 μm, about 10 nm to about 1 μm, about 10 nm to about 800 nm, about 10 nm to about 600 nm, about 10 nm to about 500 nm, about 10 nm to about 250 nm, about 50 nm to about 2 μm, about 50 nm to about 1.5 μm, about 50 nm to about 1 μm, about 50 nm to about 800 nm, about 50 nm to about 600 nm, about 50 nm to about 500 nm, about 50 nm to about 250 nm, about 100 nm to about 2 μm, about 100 nm to about 1.5 μm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

Droplet size can be determined using Brookhaven Dynamic Light Scattering (DLS). DLS is a technique used to determine droplet size in a colloidal system or emulsion. When the samples are illuminated with a monochromatic laser beam, the particles in the samples undergo Brownian motion, causing fluctuations in scattered light intensity. The scattered light is then collected at various angles, and the autocorrelation function of these intensity fluctuations is analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

### EMBODIMENTS

In a preferred embodiment, the pigment dispersion comprises, consists essentially of, or consists of:
(a) about 0.1 to about 30 wt. %, preferably about 1 to about 20 wt. %, more preferably about 5 to about 15 wt. % of a hydrophobic polymer formed as a reaction product of a natural or food-derived oil and a methacrylate or acrylate polymer, wherein preferably,
   the hydrophobic polymer is a reaction product of: (a) (i) a natural or food-derived oil selected from linseed oil, sunflower oil, tung oil, fish oil, cottonseed oil, soybean oil, or combinations thereof, preferably linseed oil, and (a) (ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly(isobutyl methacrylate);
(b) about 10 to about 90 wt. %, preferably about 25 to about 75 wt. %, more preferably about 30 to about 70 wt. % of one or more solvents capable of solubilizing the hydrophobic polymer of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa0.5 per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
   D1 is 16.8 MPa0.5,
   P1 is 4.8 MPa0.5, and
   H1 is 13.0 MPa0.5, wherein more preferably,
the one or more solvents capable of solubilizing the reaction product of (a) have a dispersion component (D), a polar component (P), and a hydrogen bonding component (H), and a distance (Ra) of less than or equal to 9.9 MPa0.5 per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
   D1 is 16.4 MPa0.5,
   P1 is 5.0 MPa0.5, and
   H1 is 11.7 MPa0.5, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 5 to about 60 wt. %, preferably about 10 to about 55 wt. %, more preferably about 35 to about 55 wt. % of particles of one or more pigments dispersed throughout the one or more solvents of (b), wherein the one or more pigments are preferably selected from inorganic pigments, organic pigments, or combinations thereof, wherein more preferably
   the one or more inorganic pigments is selected from colored metal oxides, metal hydroxides, metal oxide hydrates, silicates, metal sulfides, complex metal cyanides, metal sulfates, bronze pigments, colored mica- or mica-based pigments coated with at least one metal oxide and/or a metal oxychloride, or combinations thereof, for example, titanium dioxide, carbon black, black titanium oxide, yellow iron oxide, black iron oxide, red iron oxide, ultramarine blue, iron blue, chromium oxide, chromium hydroxide, carmine, and shikonin, activated carbon, talc, iron oxides, titanium dioxide, zinc oxide, silica, cerium oxides, zirconium oxides, aluminium oxides, calcium carbonate, barium sulfate, chromium oxides, manganese oxides, bismuth oxychloride, ultramarine, calcium sulfate, alkali magnesium silicates or mixtures thereof; and
   the one or more organic pigments are selected from nitroso, nitro-azo, xanthene, anthraquinone, isoindolinone, isoindolinone, quinacridone, perinone, perylene, diketo-pyrrolopyorrole, indigo, thioindido, dioxazine triarylmethane compounds, or mixtures thereof;
(d) optionally, one or more solid fatty compounds;
(e) optionally, one or more oil thickening agents;
(f) optionally, one or more lipophilic film-forming polymers; and
(g) optionally, one or more miscellaneous ingredients.

In another preferred embodiment, the pigment dispersion comprises, consists essentially of, or consists of:
(a) about 0.1 to about 30 wt. %, preferably about 1 to about 20 wt. %, more preferably about 5 to about 15 wt. % of a hydrophobic polymer formed as a reaction product of a natural or food-derived oil and a methacrylate or acrylate polymer, wherein preferably,
   the hydrophobic polymer is a reaction product of: (a) (i) a natural or food-derived oil selected from linseed oil, sunflower oil, tung oil, fish oil, cottonseed oil, soybean oil, or combinations thereof, preferably linseed oil, and (a) (ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly(isobutyl methacrylate);
(b) about 10 to about 90 wt. %, preferably about 25 to about 75 wt. %, more preferably about 30 to about 70 wt. % of one or more solvents capable of solubilizing the hydrophobic polymer of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
   D1 is 16.8 MPa0.5,
   P1 is 4.8 MPa0.5, and
   H1 is 13.0 MPa0.5, wherein more preferably,
the one or more solvents capable of solubilizing the reaction product of (a) have a dispersion component (D), a polar component (P), and a hydrogen bonding component (H), and a distance (Ra) of less than or equal to 9.9 MPa0.5 per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
   D1 is 16.4 MPa0.5,
   P1 is 5.0 MPa0.5, and
   H1 is 11.7 MPa0.5, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 5 to about 60 wt. %, preferably about 10 to about 55 wt. %, more preferably about 35 to about 55 wt. % of particles of one or more pigments dispersed throughout the one or more solvents of (b), wherein the one or more pigments are preferably selected from inorganic pigments, organic pigments, or combinations thereof, wherein more preferably
   the one or more inorganic pigments is selected from colored metal oxides, metal hydroxides, metal oxide hydrates, silicates, metal sulfides, complex metal cyanides, metal sulfates, bronze pigments, colored mica- or mica-based pigments coated with at least one metal oxide and/or a metal oxychloride, or combinations thereof, for example, titanium dioxide, carbon black, black titanium oxide, yellow iron oxide, black iron oxide, red iron oxide, ultramarine blue, iron blue, chromium oxide, chromium hydroxide, carmine, and shikonin, activated carbon, talc, iron oxides, titanium dioxide, zinc oxide, silica, cerium oxides, zirconium oxides, aluminium oxides, calcium carbonate, barium sulfate, chromium oxides, manganese oxides, bismuth oxychloride, ultramarine, calcium sulfate, alkali magnesium silicates or mixtures thereof; and
   the one or more organic pigments are selected from nitroso, nitro-azo, xanthene, anthraquinone, isoindolinone, isoindolinone, quinacridone, perinone, perylene, diketo-pyrrolopyorrole, indigo, thioindido, dioxazine triarylmethane compounds, or mixtures thereof, for example, carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470, and mixtures thereof;
(d) optionally, about 0.1 to about 20 wt. %, preferably about 1 to about 15 wt. %, more preferably about 1 to about 10 wt. % of one or more solid fatty compounds, preferably wherein the one or more solid fatty compounds are selected from solid animal and solid vegetable oils and fats, fully hydrogenated or partially hydrogenated vegetable and animal oils and fats, saturated fatty acids, partially hydrogenated or fully hydrogenated fatty acids, fatty acid esters, saturated, partially hydrogenated or fully hydrogenated monoglycerides, diglycerides and triglycerides, phospholipids, lecithins, partially hydrogenated or completely hydrogenated phospholipids and lecithins, lysolecithins and lysophosphatidylcholine, animal waxes, plant waxes, mineral waxes, synthetic waxes, wax esters, saturated and unsaturated fatty alcohols, fatty alcohol ethers/esters; solid saturated and unsaturated hydrocarbons (paraffins); solid silicones and silicone ethers/esters; polyol ethers/esters: glycerol ethers/esters, sorbitan, sorbitan stearate, glyceryl ricinoleate; polyglycerols and their ethers/esters, hydrophobic gelling agents: silicon dioxide, polyethylenes, or mixtures thereof;
(e) optionally, about 0.1 to about 8 wt. %, preferably about 0.5 to about 5 wt. %, more preferably from about 1 to about 3 wt. % of one or more oil thickening agents, wherein preferably the one or more oil thickening agents are selected from polymers, gums, organoclays, polyethylenes, silica, or combinations thereof, preferably wherein the one or more oil thickening agents are selected from acrylate copolymer, hectorite gel, guar gum, cellulose gum, Caesalpinia spinosa gum, gum Arabic, starch, silica, silica dimethyl silylate, behenate, glyceryl dibehenate, behenyl behenate, or mixtures thereof. In various embodiments, polyalcohol acrylates may be useful, for examples, poly C10-30 alkyl acrylate, or mixtures thereof;
(f) optionally, about 0.01 to about 10 wt. %, preferably about 0.1 to about 8 wt. %, more preferably about 0.5 to about 5 wt. % of one or more lipophilic film-forming polymers, preferably one or more lipophilic film-forming polymers selected from polybutene, alkylcelluloses with a linear or branched, saturated or unsaturated C₁ to C₈ alkyl radical, such as ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymers, silicone resins of MQ type, silsesquioxane resins, vinyl polymers grafted with a carbosiloxane dendrimer, polyalkylsiloxanes and polyarylsiloxanes, vinyl ester polymers and copolymers, vinylpyrrolidone copolymers, or mixtures thereof; and
(g) optionally, about 0.1 to about 10 wt. %, preferably about 0.5 to about 8 wt. %, more preferably about 1 to about 5 wt. % of one or more miscellaneous ingredients, preferably wherein the one or more miscellaneous ingredients are selected from preservatives, fragrances, pH adjusters, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, and mixtures thereof.

In yet another preferred embodiment, an oil-in-water emulsion comprises, consists essentially of, or consists of:
(i) an oil phase comprising, consisting essentially of, or consisting of a pigment dispersion as described in the preferred embodiments above; and
(ii) an aqueous phase comprising, consisting essentially of, or consisting of:
   (a) about 35 to about 99 wt. %, preferably about 45 to about 95 wt. %, more preferably about 50 to about 90 wt. % of water;
   (b) optionally, about 0.1 to about 30 wt. %, preferably about 0.5 to about 20 wt. %, more preferably about 1 to about 15 wt. % of one or more water soluble solvents, preferably one or more water soluble solvents selected from mono-alcohols (for example C2-8, or C2-4 alcohols), polyols (polyhydric alcohols), glycols, and a mixture thereof;
   (c) optionally, one or more water soluble dyes;
   (d) about 0.1 to about 15 wt. %, preferably about 0.5 to about 10 wt. %, more preferably about 1 to about 8 wt. % of one or more surfactants, preferably one or more biosurfactants, anionic surfactants, nonionic surfactants, amphoteric surfactants, cationic surfactants, or combinations thereof, wherein more preferably, at least one of the one or more surfactants is a biosurfactants, preferably selected from a glycolipid selected from sophorolipids, rhamnolipids, trehalose lipids, mannosylerythritol lipids, and combinations thereof, wherein preferably at least one of the one or more biosurfactants is a rhamnolipid;
   (e) optionally, about 0.1 to about 10 wt. %, preferably about 0.5 to about 8 wt. %, more preferably about 1 to about 5 wt. % of one or more miscellaneous ingredients, preferably wherein the one or more miscellaneous ingredients are selected from preservatives, fragrances, pH adjusters, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, and mixtures thereof;
      wherein the oil phase and aqueous phase are in a weight ratio of about 1:5 to about 5:1, preferably about 1:4 to about 2:1, more preferably about 1:3 to about 1:1 (oil phase: aqueous phase).

In various embodiments, the average droplet size of the oil phase in the emulsions forming the emulsions is from about 10 nm to about 2 μm, about 10 nm to about 1.5 μm, about 10 nm to about 1 μm, about 10 nm to about 800 nm, about 10 nm to about 600 nm, about 10 nm to about 500 nm, about 10 nm to about 250 nm, about 50 nm to about 2 μm, about 50 nm to about 1.5 μm, about 50 nm to about 1 μm, about 50 nm to about 800 nm, about 50 nm to about 600 nm, about 50 nm to about 500 nm, about 50 nm to about 250 nm, about 100 nm to about 2 μm, about 100 nm to about 1.5 μm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

### EXAMPLES

Implementation of the present disclosure is provided by way of the following examples. The examples serve to illustrate the technology without being limiting in nature.

### EXAMPLES

### Example 1

The following compositions were prepared by combining the pigments (TiO₂, Pigment Red 7, or Black Iron Oxide) with the caprylic/capric triglyceride using a speedmixer at 2500 rpm for 5 minutes at room temperature (23-25° C.). For compositions A, D, E, the MycelX® was dissolved in caprylic/capric triglyceride before adding the pigments. Then pigment was added and mixed at 2500 rpm for 5 minutes in the speedmixer at 2500 rpm at room temperature (23-25° C.).

| INGREDIENTS | | A | B | C | D | E | F |
|---|---|---|---|---|---|---|---|
| (a) | MycelX ®¹ | 11.5 | | 14.4 | | 11.5 | |
| (b) | TiO₂ | 50 | 50 | | 37.5 | | |
| | Red 7 | | | 37.5 | | | |
| | Black Iron Oxide | | | | | 50 | 50 |
| (c) | Caprylic/Capric | 38.5 | 50 | 48.1 | 62.5 | 38.5 | 50 |
| | Triglyceride | | | | | | |

¹Reaction product of linseed oil and isobutyl methacrylate polymer.

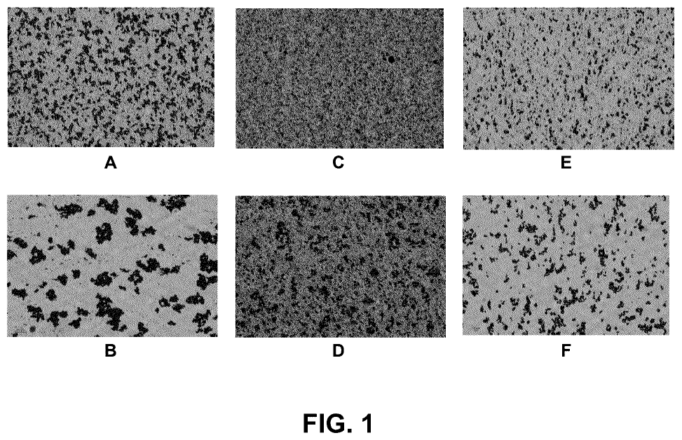

About 10 microliters of sample was dropped onto the surface of the glass slide in the center, and a cover slip was put on top without excessive force. A microscope was used to visually evaluate the dispersion of the pigments on the slide. FIG. 1 includes photographs of each composition (magnified 10×). The photographs show in that the compositions comprising MycelX® (A, C, and E) the pigments were more evenly and uniformly dispersed throughout the sample and the pigments were far less agglomerated in comparison to the compositions without the MycelX® (B, D, and F).

### Example 2

The following compositions were prepared by combining the pigment (TiO₂) with the caprylic/capric triglyceride using a speedmixer at 2500 rpm for 5 minutes at room temperature (23-25° C.). For compositions G, MycelX® was dissolved in caprylic/capric triglyceride before adding the pigment. Then pigment was added and mixed at 2500 rpm for 5 minutes in the speedmixer at 2500 rpm at room temperature (23-25° C.).

| | | |
|---|---|---|
| INGREDIENTS | G | H |

| | | | |
|---|---|---|---|
| (a) | MycelX ®¹ | 2.5 | |
| (b) | TiO₂ | 50 | 50 |
| | Red 7 | | |
| | Black Iron Oxide | | |
| (c) | Caprylic/Capric | 47.5 | 50 |
| | Triglyceride | | |

¹Reaction product of linseed oil and isobutyl methacrylate polymer.

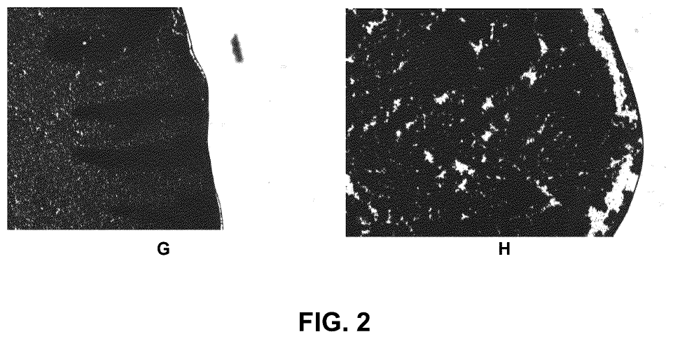

About 10 microliters of sample was dropped onto the surface of the glass slide in the center, and a cover slip was put on top without excessive force. A microscope was used to visually evaluate the dispersion of the pigment on the slide. FIG. 2 includes photographs of each composition (magnified 10×). The photographs show that the pigment (TiO₂) in composition G was evenly and uniformly disposed throughout the sample and was far less agglomerated than in composition H, without the MycelX®.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure.

The term “translucent” with respect to a translucent composition indicates that the composition has a transmittance of at least 50% at a wavelength of 600 nm, for example measured using a Lambda 40 UV-visible spectrometer.

Other than in the operating examples, or where otherwise indicated, all amount expressing quantities of ingredients and/or reaction conditions may be modified in all instances by the term “about,” meaning within +/−5% of the indicated number. Thus, for a range of “about 1 to about 10 wt. %,” The lower amount of “about 1 wt. %” may extend down to 0.95 wt. %, which is 5% less than 1 wt. %. The higher amount of “about 10 wt. %” may extend up to 10.5 wt. %, which is 5% higher than 10 wt. %, i.e., a range of “0.95 wt. % to 10.5 wt. %.”

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both oily solvent and a surfactant. If a particular composition includes both an oily solvent and a surfactant, a single compound will serve as only the oily solvent or only as the surfactant (the single compound does not simultaneously serve as both the oily solvent and the surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. For example, if a composition is essentially free from compound X, the composition includes less that 2 wt. % of compound X, or less than 1 wt. % of compound X, or less than 0.5 wt. % of compound X, or less than 0.1 wt. % of compound X, or is free from compound X.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A pigment dispersion comprising:
(a) a hydrophobic polymer formed as a reaction product of a natural or food-derived oil and a methacrylate or acrylate polymer;
(b) one or more solvents capable of solubilizing the hydrophobic polymer of (a);
(c) particles of one or more pigments dispersed throughout the one or more solvents of (b).

2. The dispersion of claim 1, wherein the hydrophobic polymer is the reaction product formed from a natural or food-derived oil and a methacrylate polymer.

3. The dispersion of claim 1, wherein the natural or food-derived oil is a drying oil or semi-drying oil selected from linseed oil, sunflower oil, tung oil, fish oil, cottonseed oil, soybean oil, or combinations thereof.

4. The dispersion of claim 1, wherein the polymer is derived from monomers selected from isobutyl methacrylate, methyl methacrylate, ethyl methacrylate, n-butyl methacrylate, and combinations thereof.

5. The dispersion of claim 1, wherein the hydrophobic polymer is the reaction product of about 50 to about 85 parts by weight of the natural or food-derived oil and about 15 to about 50 parts by weight of the methacrylate or acrylate polymer.

6. The dispersion of claim 1, wherein the hydrophobic polymer is the reaction product of about 72 to about 77 parts by weight of the natural or food-derived oil and about 23 to about 28 parts by weight of a methacrylate polymer.

7. The dispersion of claim 1, wherein the polymer is derived from one or more monomers selected from isobutyl methacrylate, methyl methacrylate, ethyl methacrylate, n-butyl methacrylate, or combinations thereof.

8. The dispersion of claim 1, wherein the polymer is isobutyl methacrylate polymer.

9. The dispersion of claim 1, wherein the hydrophobic polymer is the reaction product of linseed oil and poly(isobutyl methacrylate).

10. The dispersion of claim 1, wherein the one or more solvents capable of solubilizing the hydrophobic polymer of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):
$$Ra = \sqrt{4 {( D - {D}_{1} )}^{2} + {( P - {P}_{1} )}^{2} + {( H - {H}_{1} )}^{2}} \qquad ( I )$$
wherein
D₁ is 16.8 MPa^{0.5},
P₁ is 4.8 MPa^{0.5}, and
H₁ is 13.0 MPa^{0.5}.

10. The dispersion of claim 1, wherein at least one of the one or more solvents capable of solubilizing the hydrophobic polymer of (a) is selected from polycitronellol acetate, caprylic/capric triglyceride, isododecane, isohexadecane, tetradecane, isopropyl myristate, isopropyl alcohol, octyldodecanol, ethanol, phenoxyethanol, castor oil, or mixtures thereof.

11. The dispersion of claim 1, wherein the one or more pigments are selected from inorganic pigments, wherein the inorganic pigments are selected from metal oxides, metal borates, metal sulfates, metal sulfides, metal chromates, metal carbonates, metal selenides, and combinations thereof, more preferably, wherein the inorganic pigments are selected from metal oxides, metal hydroxides, metal oxide hydrates, silicates, metal sulfides, complex metal cyanides, metal sulfates, bronze pigments, colored mica- or mica-based pigments coated with at least one metal oxide and/or a metal oxychloride, or combinations thereof.

12. The dispersion of claim 1, wherein the one or more pigments are selected from organic pigments, wherein the organic pigments are selected from nitroso, nitro-azo, xanthene, anthraquinone, isoindolinone, isoindolinone, quinacridone, perinone, perylene, diketo-pyrrolopyorrole, indigo, thioindido, dioxazine, triarylmethane compounds, or mixtures thereof.

13. The dispersion of claim 1, wherein the one or more pigments are selected from titanium dioxide, Pigment Red 7, black iron oxide, or mixtures thereof.

14. The dispersion of claim 1, wherein the one or more pigments have a particle size of less than 15 μm.

15. A pigment dispersion comprising:
(a) about 1 to about 20 wt. % of a hydrophobic polymer formed as a reaction product of a natural or food-derived oil and a methacrylate or acrylate polymer;
(b) about 30 to about 70 wt. % of one or more solvents capable of solubilizing the hydrophobic polymer of (a); and
(c) about 30 to about 60 wt. % of particles of one or more pigments dispersed throughout the one or more solvents of (b).

16. An oil-in-water emulsion comprising:
(i) an oil phase comprising the pigment dispersion of claim 14; and
(ii) an aqueous phase comprising water.

17. The emulsion of claim 16, wherein the oil phase and the aqueous phase are in a weight ratio of about 1:4 to about 4:1.

18. A makeup product comprising the pigment dispersion of claim 1.

19. A makeup product comprising the oil-in-water emulsion of claim 16.

20. A method for making the pigment dispersion of claim 1 comprising:
(i) obtaining a hydrophobic polymer formed as a reaction product of a natural or food-derived oil and a methacrylate or acrylate polymer;
(ii) dissolving the hydrophobic polymer of (i) in one or more solvents capable of dissolving the hydrophobic polymer; and
(iii) dispersing particles of one or more pigments throughout the one or more solvents of (ii) having the hydrophobic polymer dissolved therein.
